(19) United States
(12) Patent Application Publication
(10) Pub. No.: US 2022/0415506 A1
(43) Pub. Date: Dec. 29, 2022

(54) LEARNING APPARATUS, ESTIMATION APPARATUS, LEARNING METHOD, ESTIMATION METHOD AND PROGRAM

(71) Applicant: NIPPON TELEGRAPH AND TELEPHONE CORPORATION, Tokyo (JP)

(72) Inventors: Shuhei YAMAMOTO, Tokyo (JP); Takeshi KURASHIMA, Tokyo (JP); Hiroyuki TODA, Tokyo (JP)

(73) Assignee: NIPPON TELEGRAPH AND TELEPHONE CORPORATION, Tokyo (JP)

(21) Appl. No.: 17/779,179

(22) PCT Filed: Nov. 27, 2019
(86) PCT No.: PCT/JP2019/046409

§ 371 Date: May 24, 2022

(51) Int. Cl. G16H 50/20; G06N 3/08

(52) U.S. Cl. CPC G16H 50/20; G06N 3/08

(57) ABSTRACT

A training apparatus includes a feature extraction unit that extracts feature vector data from behavior data of each date and time, a reference point extraction unit that performs, for each date and time, processing of calculating a difference between feature vector data of certain date and time and each of one or more pieces of feature vector data in past within a predetermined period from the date and time, and extracting one or more pieces of difference vector data corresponding to the feature vector data of the date and time, and a state estimation model training unit that trains a state estimation model using feature vector data of each date and time, difference vector data, and state information.

### TECHNICAL FIELD

The present invention relates to a technique for estimating a psychological state of the user from behavior data of the user.

### BACKGROUND ART

With the spread of wearable sensors represented by smartwatches, fitness trackers, smartphones, and the like, it has become possible to easily record biometric information and behavior logs of the user. Hereinafter, biometric information and behavior logs are collectively referred to as behavior data.

Detailed analysis of behavior data and a psychological state (degree of happiness, degree of stress, degree of health, and the like) obtained by self-evaluation of the user is useful for various applications. For example, if the history of the user's biometric information acquired through a smartwatch can be used to estimate the present day stress level as a numerical value or predict future health level as a numerical value, it will be effective in improving the user's psychological state. It is useful for various purposes such as recommending various actions.

In the related art, as a technique for automatically estimating a psychological state of the user from such behavior data, there is a technique for discretizing obtained data, converting the data into a histogram, and estimating degree of health and degree of stress by a probabilistic generative model (NPL 1). Further, development of a technique for predicting a future psychological state by deep learning using an operation log and daily series data of screen time acquired from a smartphone has also been worked on (NPL 2).

### CITATION LIST

### Non Patent Literature

NPL 1: E. Nosakhare and R. Picard: Probabilistic Latent Variable Modeling for Assessing Behavioral Influences on Well-Being. In Proc. of KDD, 2019.

NPL 2: D. Spatis, S. Servia-Rodriguez, K. Farrahi, C. Mascolo, and J. Rentflow: Sequence Multi-task Learning to Forecast Mental Wellbeing from Sparse Self-reported Data. In Proc. of KDD, 2019.

### SUMMARY OF THE INVENTION

### Technical Problem

However, in the above related-art method, since data is separated and processed on a daily basis, it has been not possible to consider a difference in self-evaluation of the user due to time-series fluctuations depending on date and time. For example, even in a state where a body temperature is higher than that in normal time, in a case where the body temperature is higher than that on the previous day, the user feels that the physical condition is bad and self-evaluates as such, and in a case where the body temperature is lower than that on the previous day, the user feels that the physical condition is better than that on the previous day and self-evaluates as such.

As a result, there is a difference in results of self-evaluation even for the same body temperature. However, the related-art method hardly automatically estimate this. Even in other situations, the user compares his or her own psychological state with that at a past reference point at time intervals such as last weeks and last months. For this reason, the above is a problem that may occur for various indices of psychological states. Further, there is a problem that which reference point the self-evaluation is based on differs depending on the user and an index to be evaluated. Further, this problem may occur not only in estimation of a psychological state of the user, but also in entire estimation of a state obtained by self-evaluation of the user.

In view of the above-described problem of the related art, an object of the present invention is to provide a technique that enables estimation of a state felt by the user with high accuracy based on behavior data of the user.

### Means for Solving the Problem

According to a disclosed technique, there is provided a training apparatus including a feature extraction unit configured to extract feature vector data from behavior data of each date and time, a reference point extraction unit configured to perform, for each date and time, processing of calculating a difference between feature vector data of certain date and time and each of one or more pieces of feature vector data in past within a predetermined period from the date and time, and extracting one or more pieces of difference vector data corresponding to the feature vector data of the date and time, and a state estimation model training unit configured to train a state estimation model using feature vector data of each date and time, difference vector data, and state information.

### Effects of the Invention

According to the disclosed technique, there is provided a technique capable of estimating a state felt by the user with high accuracy based on behavior data of the user.

### BRIEF DESCRIPTION OF DRAWINGS

FIG. 1 is a configuration diagram of a training apparatus.

FIG. 2 is a configuration diagram of an estimation apparatus.

FIG. 3 is a diagram illustrating an example of a hardware configuration of the apparatuses.

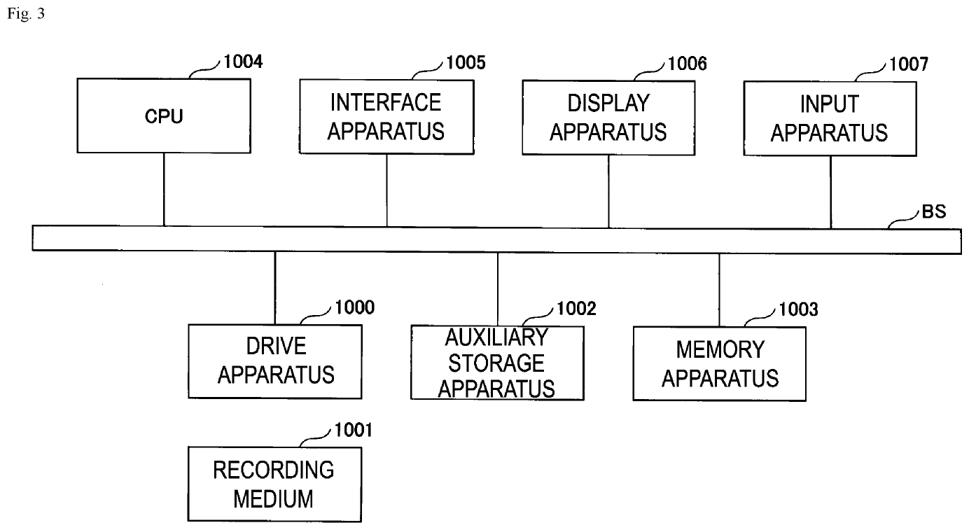

FIG. 4 is a flowchart illustrating operation of the training apparatus.

FIG. 5 is a flowchart illustrating operation of the estimation apparatus.

FIG. 6 is a diagram illustrating an example of a storage format of a behavior data DB.

FIG. 7 is a diagram illustrating an example of feature vector data obtained as processing of a feature extraction unit.

FIG. 8 is a diagram illustrating an example of difference vector data obtained as processing of a reference point extraction unit.

FIG. 9 is a diagram illustrating an example of a storage format of a self-evaluation data DB.

FIG. 10 is a diagram illustrating an example of a storage format of an estimated parameter storage DB.

FIG. 11 is a flowchart illustrating operation of a behavior data preprocessing unit.

FIG. 12 is a flowchart illustrating operation of the behavior data preprocessing unit.

FIG. 13 is a flowchart illustrating operation of the reference point extraction unit.

FIG. 14 is a diagram illustrating an example of a DNN constructed by a psychological state estimation model construction unit.

FIG. 15 is a flowchart illustrating operation of a psychological state estimation model training unit.

FIG. 16 is a diagram illustrating an example of a storage format of a psychological state estimation DNN model DB.

FIG. 17 is a flowchart illustrating operation of a psychological state estimation unit.

FIG. 18 is a flowchart illustrating operation of an estimation result visualization unit.

FIG. 19 is a diagram illustrating an example of an estimation result visualization method.

### DESCRIPTION OF EMBODIMENTS

Hereinafter, embodiments of the present disclosure will be described with reference to the drawings. The embodiment to be described below is merely exemplary, and an embodiment to which the present invention is applied is not limited to the following embodiment. For example, in the embodiment described below, an example in which a psychological state of the user is estimated from behavior data is described. However, the present invention can also be applied to estimating a state (for example, a fatigue state) sensed by the user other than a psychological state.

### Apparatus Configuration

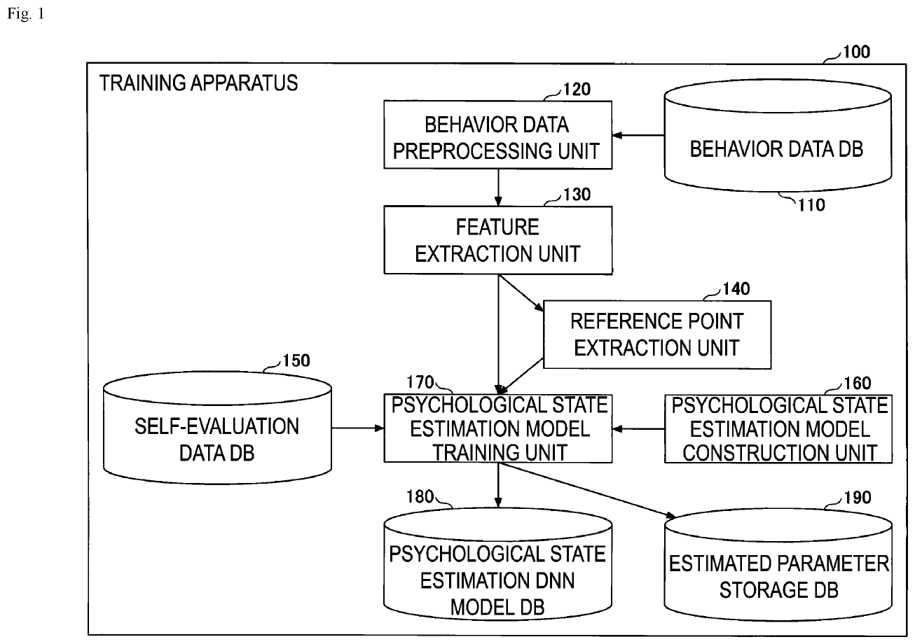

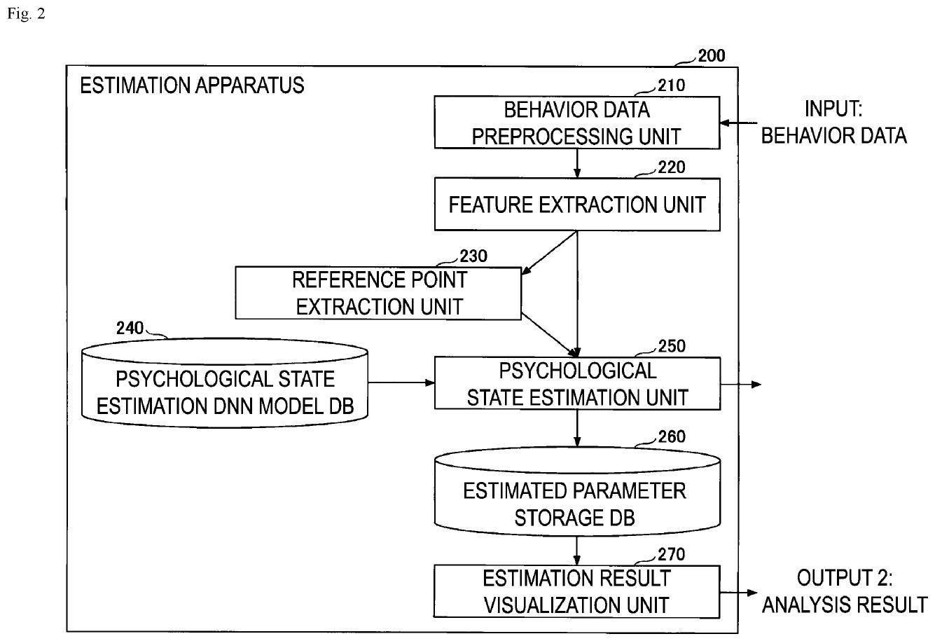

FIG. 1 illustrates a configuration of a training apparatus 100 that performs processing of a training phase for psychological state estimation according to an embodiment of the present invention. FIG. 2 illustrates a configuration of an estimation apparatus 200 that performs processing of an estimation phase for psychological state estimation. Note that an apparatus including both a function of the training apparatus 100 and a function of the estimation apparatus 200 may be provided. The apparatus may be referred to as a psychological state estimation apparatus. Further, a psychological state estimation apparatus including both the function of the training apparatus 100 and the function of the estimation apparatus 200 may be referred to as a training apparatus, or may be referred to as an estimation apparatus.

As illustrated in FIG. 1, the training apparatus 100 includes a behavior data DB (Database) 110, a behavior data preprocessing unit 120, a feature extraction unit 130, a reference point extraction unit 140, a self-evaluation data DB 150, a psychological state estimation model construction unit 160, a psychological state estimation model training unit 170, a psychological state estimation DNN (Deep Neural Network) model DB 180, and an estimated parameter storage DB 190.

Note that a function including the behavior data preprocessing unit 120 and the feature extraction unit 130 may be referred to as a feature extraction unit. Further, the psychological state estimation model training unit 170 may be referred to as a state estimation model training unit. Further, the training apparatus 100 may not to include the psychological state estimation model construction unit 160. In this case, a model (DNN) constructed outside the training apparatus 100 is input to the psychological state estimation model training unit 170 of the training apparatus 100.

The training apparatus 100 outputs a psychological state estimation DNN model and a parameter specific to the user clarified at the time of training using information of each DB. The parameter is, for example, a parameter indicating importance at each date and time before certain date and time regarding a psychological state of the user at the certain date and time.

The behavior data DB 110, the self-evaluation data DB 150, and the estimated parameter storage DB 190 in the training apparatus 100 are assumed to be constructed in advance so that pieces of related data are associated with a data ID. Further, the self-evaluation data DB 150 stores a numerical value of a psychological state that is self-evaluated by the user for each data ID.

Here, as the psychological state, for example, degree of happiness, stress, and the like felt by the user on that day are assumed, and text and a numerical value indicating them are assumed. For the construction processing of the self-evaluation data DB 150, for example, a self-evaluation result of a psychological state for each data ID is preferably input by the target user, and the input result is preferably stored in the DB.

As illustrated in FIG. 2, the estimation apparatus 200 includes a behavior data preprocessing unit 210, a feature extraction unit 220, a reference point extraction unit 230, a psychological state estimation DNN model DB 240, a psychological state estimation unit 250, an estimated parameter storage DB 260, and an estimation result visualization unit 270. Note that a function including the behavior data preprocessing unit 210 and the feature extraction unit 220 may be referred to as a feature extraction unit. Further, the psychological state estimation unit 250 may be referred to as a state estimation unit.

The estimation apparatus 200 outputs an estimation result for input behavior data and a parameter obtained at the time of the estimation as an analysis result. The parameter is, for example, a parameter indicating importance at each date and time before certain date and time regarding a psychological state of the user at the certain date and time.

### Example of Hardware Configuration

All of the training apparatus 100, the estimation apparatus 200, and the apparatus including both the function of the training apparatus 100 and the function of the estimation apparatus 200 can be realized by causing a computer to execute a program describing processing content described in an embodiment. Note that the “computer” may be a virtual machine provided by a cloud service. In a case where a virtual machine is used, the “hardware” described here is virtual hardware.

The apparatus (the training apparatus 100, the estimation apparatus 200, or the apparatus including both the function of the training apparatus 100 and the function of the estimation apparatus 200) can be realized by execution of a program corresponding to processing executed by the apparatus using hardware resources such as a CPU and a memory built in a computer. The program can be recorded on a computer-readable recording medium (a portable memory or the like) to be stored or distributed. The program can also be provided via a network such as the Internet or an electronic mail.

FIG. 2 is a diagram illustrating an example of a hardware configuration of the above-described computer in the present embodiment. The computer in FIG. 2 includes a drive apparatus 1000, an auxiliary storage apparatus 1002, a memory apparatus 1003, a CPU 1004, an interface apparatus 1005, a display apparatus 1006, an input apparatus 1007, and the like which are connected to each other through a bus B.

A program for implementing processing in the computer is provided by, for example, a recording medium 1001 such as a CD-ROM or a memory card. When the recording medium 1001 that stores a program is set in the drive apparatus 1000, the program is installed in the auxiliary storage apparatus 1002 from the recording medium 1001 via the drive apparatus 1000. Here, the program may not necessarily be installed from the recording medium 1001 and may be downloaded from another computer via a network. The auxiliary storage apparatus 1002 stores the installed program and also stores necessary files, data, and the like.

The memory apparatus 1003 reads the program from the auxiliary storage apparatus 1002 and stores the program in a case where an instruction to start the program is given. The CPU 1004 realizes a function related to the apparatus in the video distribution system according to the program stored in the memory apparatus 1003. The interface apparatus 1005 is used as an interface for connection to a network. The display apparatus 1006 displays a graphical user interface (GUI) or the like according to a program. The input apparatus 1007 is constituted by a keyboard, a mouse, buttons, a touch panel, or the like, and is used to input various operation instructions.

Hereinafter, operation of the training apparatus 100 and the estimation apparatus 200 will be described in detail.

### Overall Operation of Training Apparatus 100

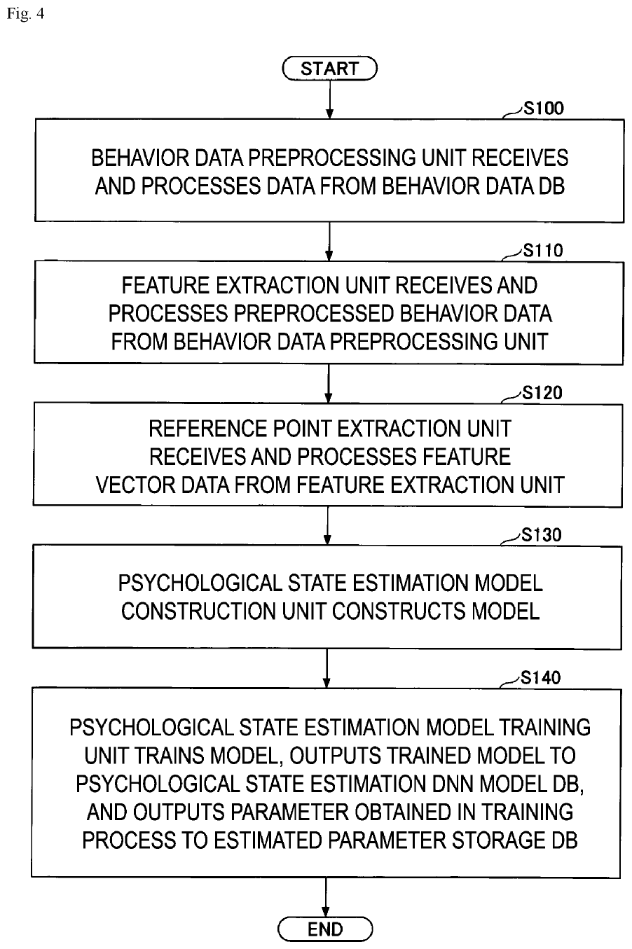

With reference to FIG. 4, the overall operation of the training apparatus 100 according to the present embodiment will be described.

### S100

In S100, the behavior data preprocessing unit 120 receives data from the behavior data DB 110 and performs preprocessing. Details of the processing will be described later.

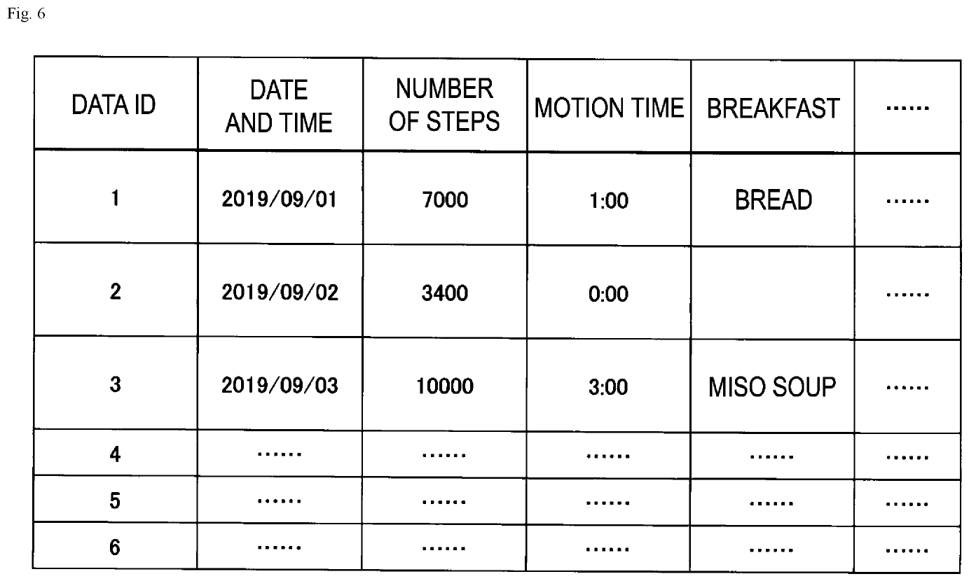

FIG. 6 illustrates an example of a storage format of data of the behavior data DB 110. As illustrated in FIG. 6, the behavior data is recorded in the form of a numerical value, a time of day, a character string, and the like in a column representing each behavior, and is associated with a data ID for association with self-evaluation data as described above. Note that the “date and time” is the date and time at which relevant behavior data is recorded. The “date and time” may be year/month/day/hour/minute/second information, may be year/month/day information, or may be other information. The “date and time” may be any information indicating when the behavior data is acquired.

### S110

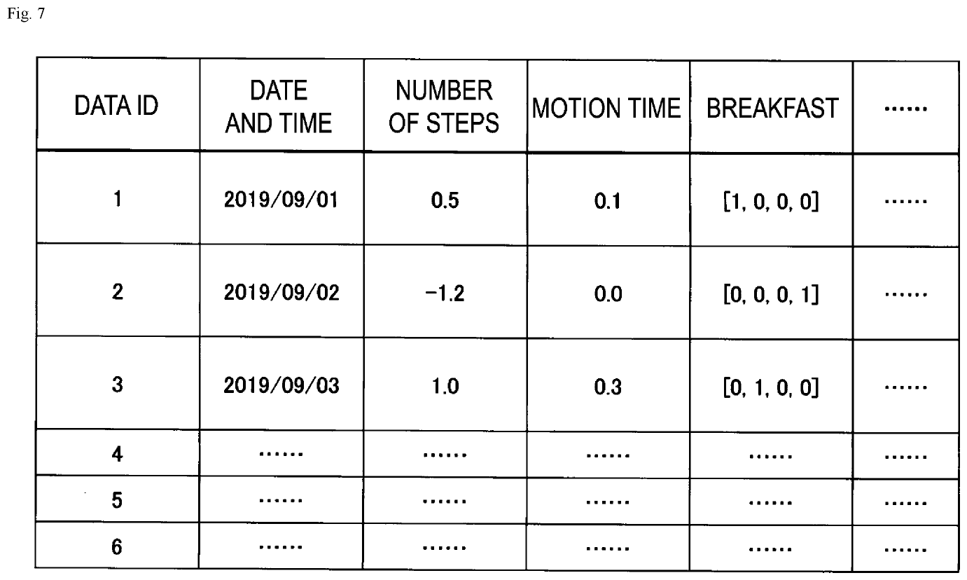

In S110, the feature extraction unit 130 receives behavior data that is preprocessed from the behavior data preprocessing unit 120 and performs feature extraction processing. Details of the processing will be described later. FIG. 7 illustrates an example of feature vector data obtained as an output of the feature extraction unit 130.

### S120

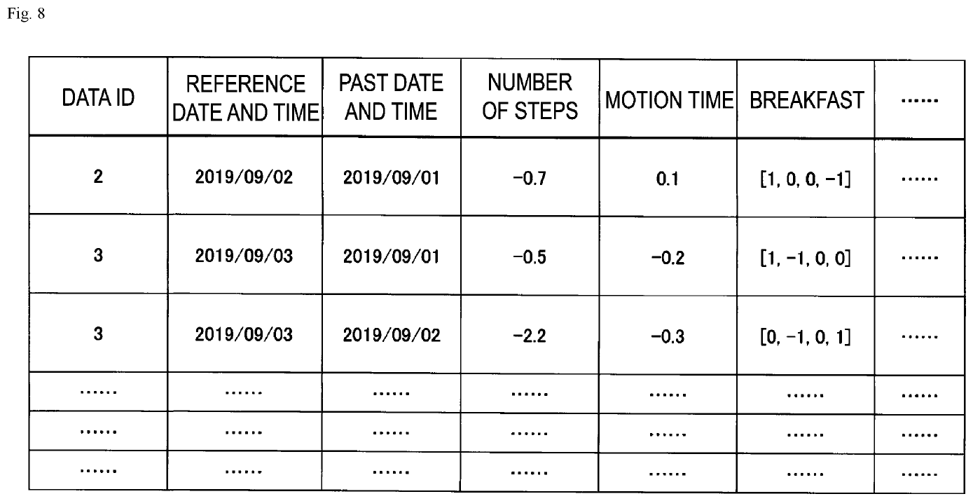

In S120, the reference point extraction unit 140 receives and processes feature vector data from the feature extraction unit 130. Details of the processing will be described later. FIG. 8 illustrates an example of difference vector data obtained as an output of the reference point extraction unit 140.

### S130

In S130, the psychological state estimation model construction unit 160 constructs a model. Details of the processing will be described later.

### S140

In S140, the psychological state estimation model training unit 170 receives feature vector data from the feature extraction unit 130, difference vector data from the reference point extraction unit 140, self-evaluation data from the self-evaluation data DB 150, and a DNN model from the psychological state estimation model construction unit 160, trains a model, outputs a trained model to the psychological state estimation DNN model DB 180, and outputs a parameter obtained in a training process to the estimated parameter storage DB 190.

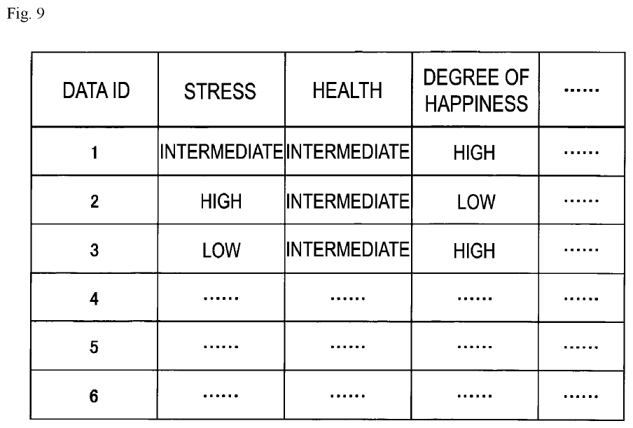

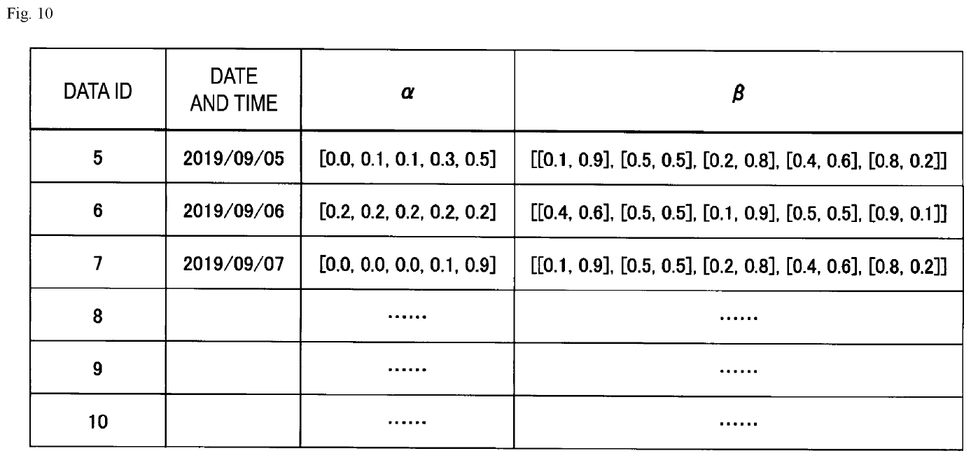

FIG. 9 illustrates an example of a storage format of the self-evaluation data DB 150, and FIG. 10 illustrates an example of a storage format of the estimated parameter storage DB 190.

### Overall Operation of Estimation Apparatus 200

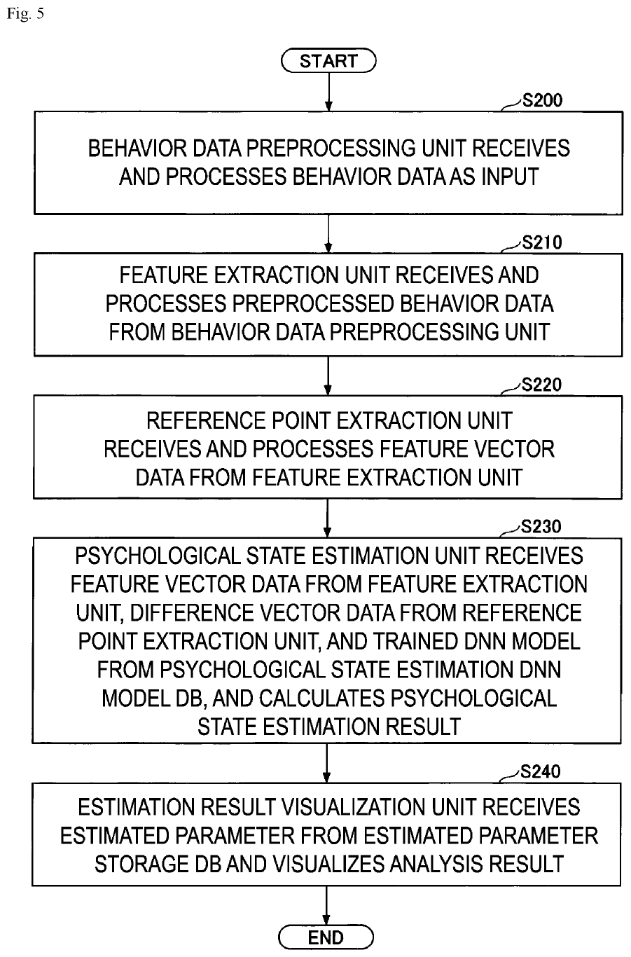

Overall operation of the estimation apparatus 200 according to the present embodiment will be described with reference to FIG. 5. As a premise of operation below, the psychological state estimation DNN model DB 240 stores a trained DNN model trained by the training apparatus 100.

### S200

In S200, the behavior data preprocessing unit 210 receives behavior data as input and performs preprocessing. A format of the behavior data is similar to the storage format of data of the behavior data DB 110 shown in FIG. 6.

### S210

In S210, the feature extraction unit 220 receives the preprocessed behavior data from the behavior data preprocessing unit 210, and performs feature extraction processing. A format of feature vector data obtained in the feature extraction processing of S210 is similar to the format of the feature vector data shown in FIG. 7.

### S220

In S220, the reference point extraction unit 230 receives and processes feature vector data from the feature extraction unit 220. A format of difference vector data obtained by the processing of S220 is similar to the format of the difference vector data illustrated in FIG. 8.

### S230

In S230, the psychological state estimation unit 250 receives feature vector data from the feature extraction unit 220, receives difference vector data from the reference point extraction unit 230, receives a trained DNN model from the psychological state estimation DNN model DB 240, and calculates and outputs a psychological state estimation result.

### S240

In S240, the estimation result visualization unit 270 receives an estimated parameter from the estimated parameter storage DB 260 and visualizes an analysis result. Details of the processing will be described later.

Hereinafter, each of the operations described above will be described more in detail.

### Detailed Operation of Behavior Data Preprocessing Unit 120 and Behavior Data Preprocessing Unit 210

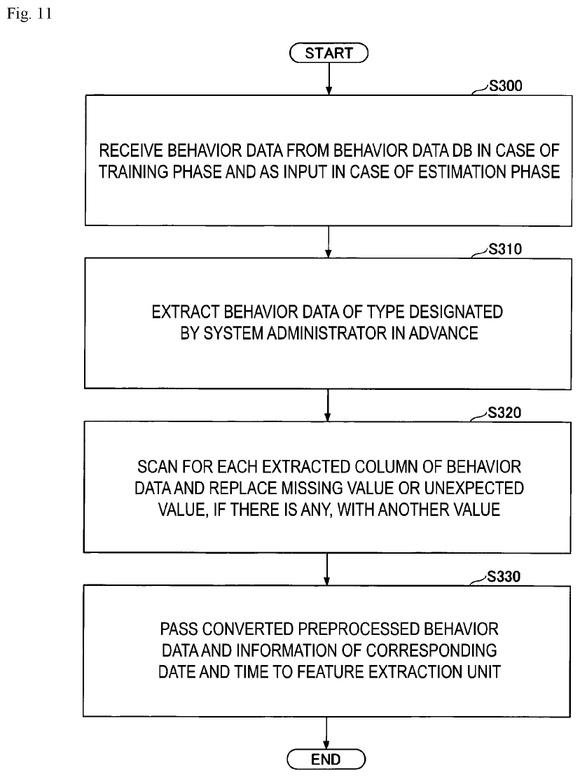

FIG. 11 is a flowchart illustrating operation of the behavior data preprocessing unit 120 and the behavior data preprocessing unit 210. Operation of the behavior data preprocessing unit 120 and the behavior data preprocessing unit 210 will be described with reference to FIG. 11. In a case where operation is common between the behavior data preprocessing unit 120 of the training apparatus 100 and the behavior data preprocessing unit 210 of the estimation apparatus 200, description will be given as “behavior data preprocessing unit 120/210”. The same applies to other functional units.

### S300

In S300, in a training phase, the behavior data preprocessing unit 120 of the training apparatus 100 acquires behavior data from the behavior data DB 110. In an estimation phase, the behavior data preprocessing unit 210 of the estimation apparatus 200 receives behavior data as input.

### S310

In S310, the behavior data preprocessing unit 120/210 extract behavior data (columns) of a type previously designated by a system administrator. For example, a column name of behavior data to be extracted is defined, and data in a column matching the column name is extracted. In the example illustrated in FIG. 6, for example, if data to be extracted is the number of steps and breakfast, data of the number of steps and breakfast are extracted.

### S320

In S320, the behavior data preprocessing unit 120/210 scan for each extracted column of behavior data, and replace a missing value or an unexpected value, if there is any, with another value. For example, an average value of a corresponding column or zero are inserted in a case of numerical data, and a character string indicating a missing value is inserted in a case of a character string type data.

### S330

In S330, the behavior data preprocessing unit 120/210 pass converted preprocessed behavior data and information of corresponding date and time to the feature extraction unit 130/220.

### Detailed Operation of Feature Extraction Unit 130 and Feature Extraction Unit 220

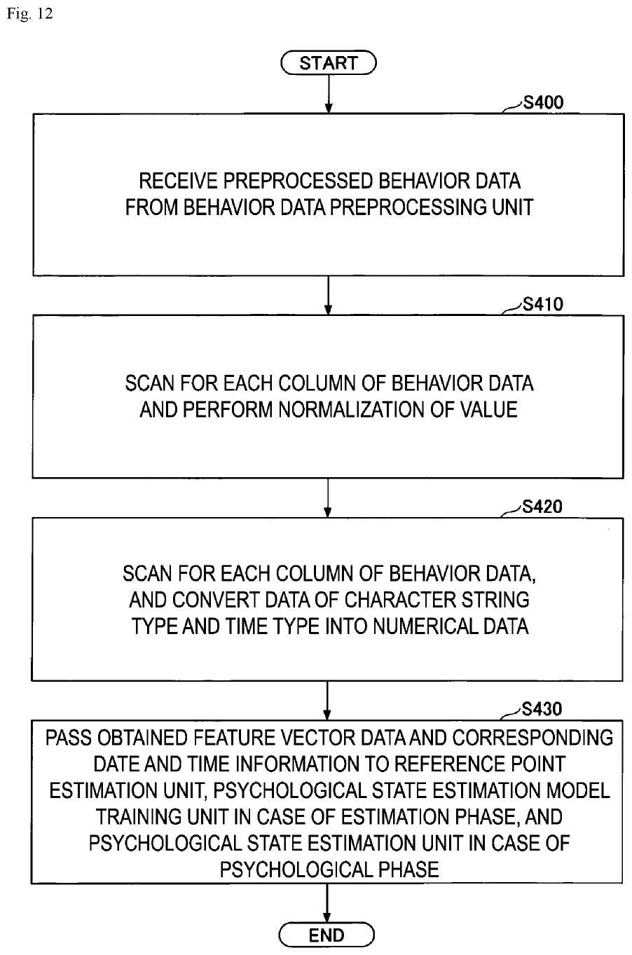

FIG. 12 is a flowchart illustrating operation of the feature extraction unit 130 and the feature extraction unit 220. The operation of the feature extraction unit 130 and the feature extraction unit 220 will be described with reference to FIG. 12.

### S400

In S400, the feature extraction unit 130/220 receive preprocessed behavior data from the behavior data preprocessing unit 120/210.

### S410

In S410, the feature extraction unit 130/220 scans for each column of behavior data, and performs normalization of a value. For example, in a case of numerical data, the data is normalized to an average of zero and a standard deviation of one, and, in a case of character string type data, the number of times that the same value appears in the whole data is counted and substituted. As a result, feature vector data for each date and time, as shown in FIG. 7, is obtained.

### S420

In S420, the feature extraction unit 130/220 scans for each column of behavior data, and converts data of a character string type and a time type into numerical data. For example, data of a character string type is converted into a vector of one-hot representation relating to a corresponding dimension, and data of a time type is converted into UNIX (trade name) time.

### S430

In S430, in a training phase, the feature extraction unit 130 of the training apparatus 100 passes obtained feature vector data and corresponding date and time information to the reference point extraction unit 140 and the psychological state estimation model training unit 170. In an estimation phase, the feature extraction unit 220 of the estimation apparatus 200 passes obtained feature vector data and corresponding date and time information to the reference point extraction unit 230 and the psychological state estimation unit 250.

### Detailed Operation of Reference Point Extraction Unit 140 and Reference Point Extraction Unit 230

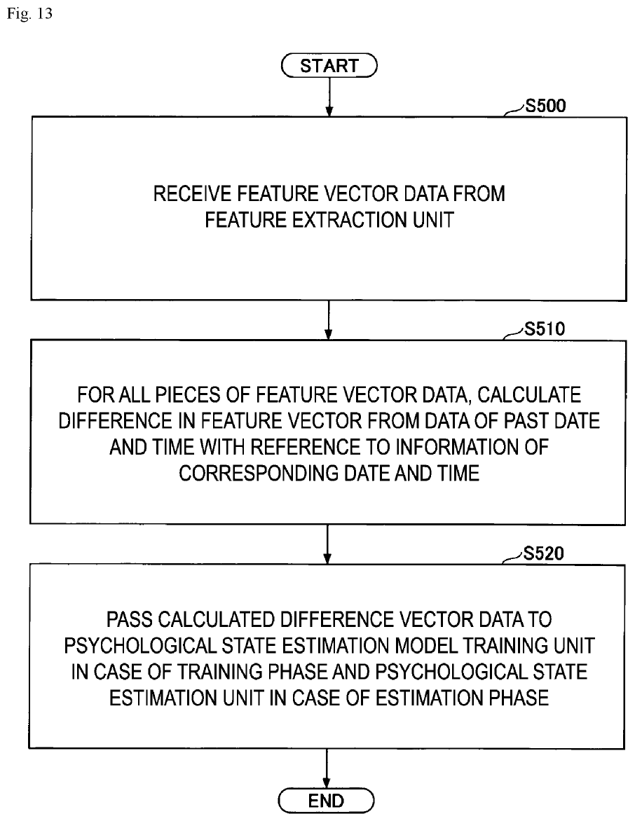

FIG. 13 is a flowchart illustrating operation of the reference point extraction unit 140 and the reference point extraction unit 230. The operation of the reference point extraction unit 140 and the reference point extraction unit 230 will be described with reference to FIG. 13.

### S500

In S500, the reference point extraction unit 140/230 receive feature vector data from the feature extraction unit 130/220.

### S510

In S510, for all pieces of feature vector data, the reference point extraction unit 140/230 calculates a difference in a feature vector from data of past date and time with reference to information of corresponding date and time, and outputs difference vector data. For example, a system administrator defines a retrospective period, such as one month before or one week before, in advance, and a difference from feature vector data corresponding to the above period is calculated for each piece of feature vector data.

Here, the unit of date and time (time) is set to 1, and feature vector data of date and time t (time t) is denoted as xₜ. For feature vector data (xₜ) of the date and time t (example of the “reference date and time” in FIG. 8), when “5” is a retrospective period, difference vector data for the feature vector data (xₜ) of the date and time t is (xₜ₋₅−xₜ, xₜ₋₄−xₜ, xₜ₋₃−xₜ, xₜ₋₂−xₜ, xₜ₋₁−xₜ, xₜ−xₜ).

In a training phase, difference vector data for each t is extracted. In an estimation phase, for example, when date and time to be estimated is one date and time S, difference vector data for S is extracted.

### S520

In S520, the reference point extraction unit 140 of the training apparatus 100 passes calculated difference vector data to the psychological state estimation model training unit 170. The reference point extraction unit 230 of the estimation apparatus 200 passes calculated difference vector data to the psychological state estimation unit 250.

### Psychological State Estimation Model Construction Unit 160

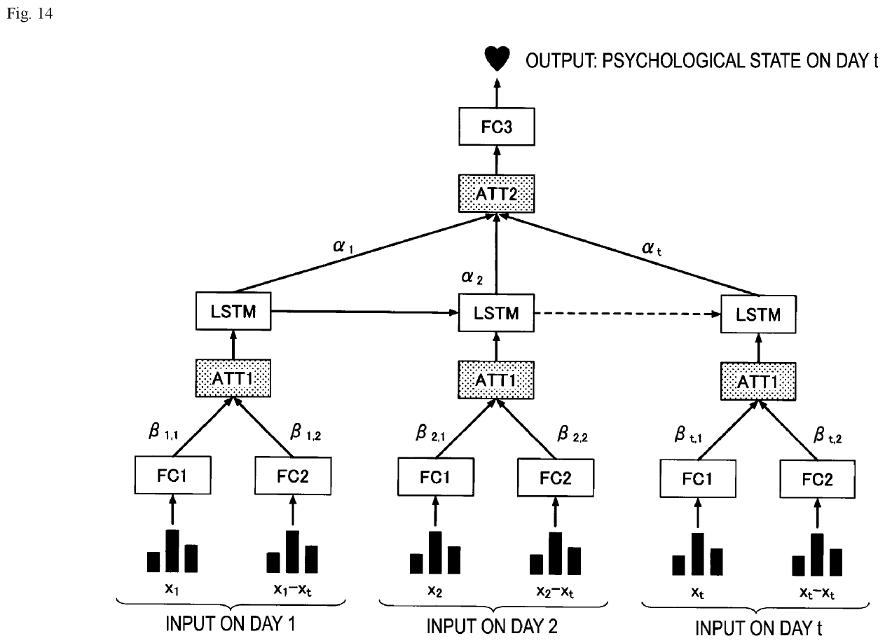

FIG. 14 is an example of a structure of a DNN constructed by the psychological state estimation model construction unit 160 of the training apparatus 100. The DNN receives, as an input, feature vector data of corresponding date and time and difference vector data, and outputs psychological state probability of the user and a parameter at the time of estimation.

The DNN has each one of a total binding layer FC1, a total binding layer FC2, a self-attention mechanism (Self-attention) ATT1, LSTM (Long-short term memory), a self-attention mechanism ATT2, and a total binding layer FC3. In an example illustrated in FIG. 14, feature vector data and difference vector data are sequentially input as input on a first day, input on a second day, and the like.

The total binding layer FC1 extracts a more abstract feature from feature vector data. The total binding layer FC1 uses a sigmoid function, the ReLu function, or the like, for example, to non-linear-transform input feature vector data to obtain a feature vector e_{t, 1}. t is an index related to date and time information.

The total binding layer FC2 extracts a more abstract feature from difference vector data. The total binding layer FC2 performs non-linear transformation processing like the total binding layer FC1 to obtain a feature vector e_{t, 2}.

The self-attention mechanism ATT1 calculates a weighted average to obtain a feature vector that takes into account the importance of two types of abstracted feature vectors (e_{t, 1}, e_{t, 2}). Calculation of weights β_{t, 1}, β_{t, 2} is achieved by two total binding layers in the self-attention mechanism ATT1.

A first total binding layer of two total binding layers outputs a context vector of any size for e_{t, k} as input, and a second total binding layer outputs a scalar value corresponding to importance β_{t, k} for a context vector as input. The context vector may undergo non-linear transformation.

The importance β_{t, k} is normalized to a value equal to or more than zero using, for example, an exponential function and the like. After normalization processing, the value may be converted to a value corresponding to a probability value by, for example, the softmax function or the like.

The LSTM further abstracts an abstracted feature vector as series data. Specifically, for example, feature vectors are sequentially received as series data, and non-linear transformation is performed repeatedly in consideration of past abstracted information.

The self-attention mechanism ATT2 obtains a feature vector in consideration of degree of importance of each date and time for a series feature vector {hₜ}^{T}ₜ₌₁ (T is reference date and time) abstracted by the LSTM. Note that {hₜ}^{T}ₜ₌₁ refers to {h₁, h₂, . . . , h_{T}}. A weight {αₜ}^{T}ₜ₌₁ corresponding to importance of each feature vector is obtained by two total binding layers in the self-attention mechanism ATT2, like the self-attention mechanisms ATT1. A first total binding layer of two total binding layers outputs a context vector of any size for hₜ as input, and a second total binding layer outputs a scalar value corresponding to importance αₜ for a context vector as input. The context vector may undergo non-linear transformation.

The total binding layer FC3 converts a feature vector weight averaged by the self-attention mechanism ATT2 into a vector of dimensions as many as types of psychological states of the target user and calculates a probability vector for each psychological state. Here, a softmax function or the like is used to perform non-linear transformation such that the sum of all elements (for example, low, medium, high) of a characteristic as output becomes one.

As described above, the DNN comprises two self-attention mechanisms for indexing degree of consideration of a reference point of the user.

Assume that the configuration of FIG. 14 has been trained. For example, in a case of use in an estimation phase for estimating a psychological state of date and time t (in unit of days), as illustrated in FIG. 14, feature vector data x₁ on the day “1”, which is a day in the past by a predetermined period from t, is input to the total binding layer FC1, and difference vector data (x₁−xₜ) is input to the total binding layer FC2. Similarly, feature vector data and difference vector data for each day to t are input to the total binding layer FC1 and the total binding layer FC2 and a psychological state from a t-th day is output from the FC3. Further, α₁ to αₜ are acquired and output.

In a training phase, feature vector data and difference vector data are input for various t, and training progresses by comparing output for t to correct psychological state data (annotation data) at t.

### Detailed Operation of Psychological State Estimation Model training unit 170

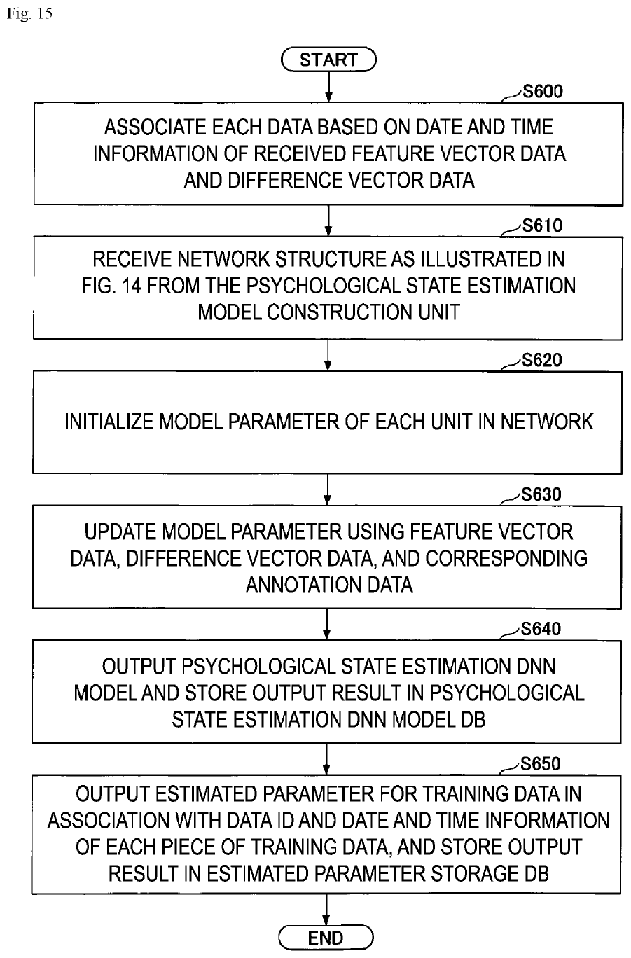

FIG. 15 is a flowchart showing operation of the psychological state estimation model training unit 170 of the training apparatus 100. Operation of the psychological state estimation model training unit 170 will be described with reference to FIG. 15.

### S600

In Step 600, the psychological state estimation model training unit 170 associates each piece of data based on date and time information of feature vector data received from the feature extraction unit 130 and date and time information of difference vector data received from the reference point extraction unit 140. To associate is, for example, in the example shown in FIG. 14, feature vector data x₁ and difference vector data (x₁−xₜ) are associated, and feature vector data x₂ and difference vector data (x₂−xₜ) are associated, and so on.

### S610

In S610, the psychological state estimation model training unit 170 receives a network structure as illustrated in FIG. 14 from the psychological state estimation model construction unit 160.

### S620

In S620, the psychological state estimation model training unit 170 initializes a model parameter of each unit in a network. Initialization is performed with a random number from 0 to 1, for example.

### S630

In S630, the psychological state estimation model training unit 170 updates a model parameter using feature vector data, difference vector data, and corresponding annotation data. Note that the annotation data is data obtained from the self-evaluation data DB 150.

### S640

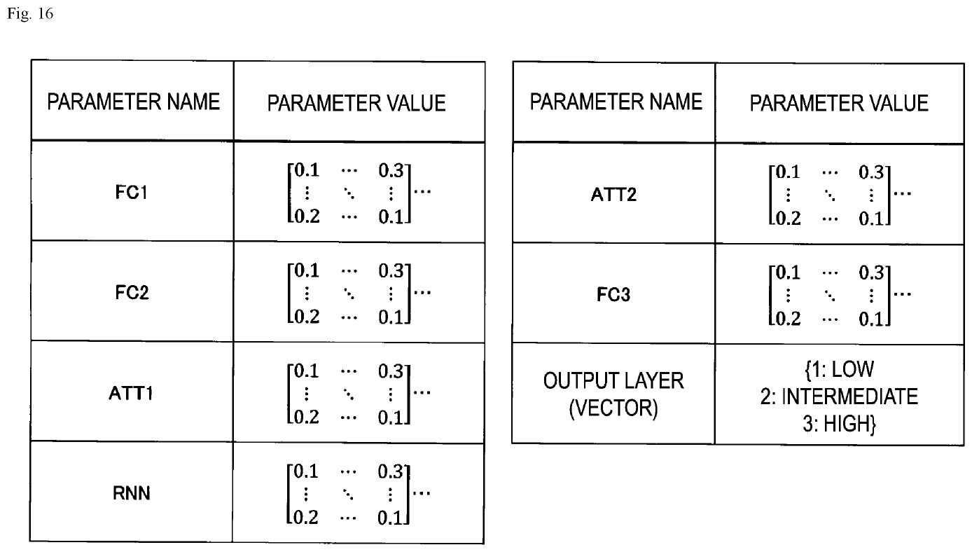

In S640, the psychological state estimation model training unit 170 outputs a trained psychological state estimation DNN model (network structure and model parameter) and stores an output result in the psychological state estimation DNN model DB 180. An example of the model parameter is described in FIG. 16. A parameter is stored as a matrix and a vector in each layer. Further, for an output layer, text of a psychological state corresponding to each element number of a probability vector is stored.

### S650

In S650, the psychological state estimation model training unit 170 outputs an estimated parameter {αₜ}^{T}ₜ₌₁ and {β_{t, 1}, β_{t, 2}}^{T}ₜ₌₁ for training data in association with a data ID and date and time information of each piece of training data, and stores an output result in the estimated parameter storage DB 190. An example of the estimated parameter is as shown in FIG. 10.

### Detailed Operation of Psychological State Estimation Unit 250

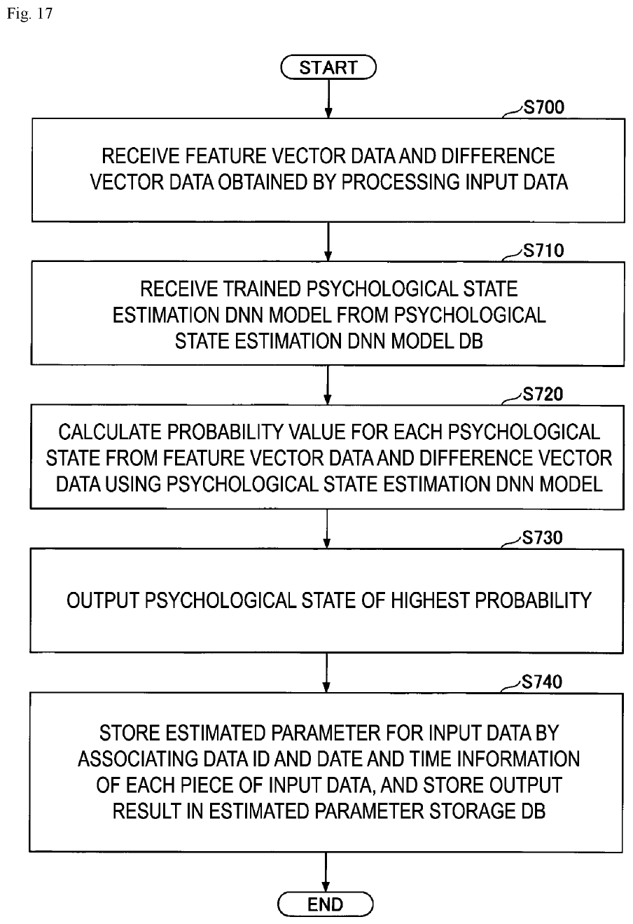

FIG. 17 is a flowchart illustrating operation of the psychological state estimation unit 250 of the estimation apparatus 200 in an estimation phase. Operation of the psychological state estimation unit 250 will be described with reference to FIG. 17. As a premise here, the psychological state estimation DNN model DB 240 stores a trained psychological state estimation DNN model trained by the training apparatus 100.

### S700

In S700, the psychological state estimation unit 250 receives, from the feature extraction unit 220, feature vector data obtained by processing of input data, and receives difference vector data from the reference point extraction unit 230.

### S710

In S710, the psychological state estimation unit 250 receives a trained psychological state estimation DNN model from the psychological state estimation DNN model DB 240.

### S720

In S720, the psychological state estimation unit 250 inputs feature vector data and difference vector data into the psychological state estimation DNN model. The psychological state estimation DNN model calculates and outputs a probability value for each psychological state.

### S730

In S730, the psychological state estimation unit 250 outputs a psychological state of a highest probability for each psychological state output from the psychological state estimation DNN model. For example, in a case where “stress” is a target, when probability that “stress” is low is 0.1, probability that “stress” is middle is 0.2, and probability that “stress” is high is 0.7, information indicating that “stress” is high is output.

### S740

In S740, the psychological state estimation unit 250 outputs a parameter {═ₜ}^{T}ₜ₌₁ and {β_{t, 1}, β_{t, 2}}^{T}ₜ₌₁ estimated for input data in association with a data ID and date and time information of the input data, and stores an output result in the estimated parameter storage DB 260. A format of the data to be stored is as shown in FIG. 10.

For example, when date and time information is T and a data ID corresponding to the date and time information is ID_T, the psychological state estimation unit 250 outputs ID_T, T, {αₜ}^{T}ₜ₌₁, {β_{t, 1}, β_{t, 2}}^{T}ₜ₌₁.

### Detailed Operation of Estimation Result Visualization Unit 270

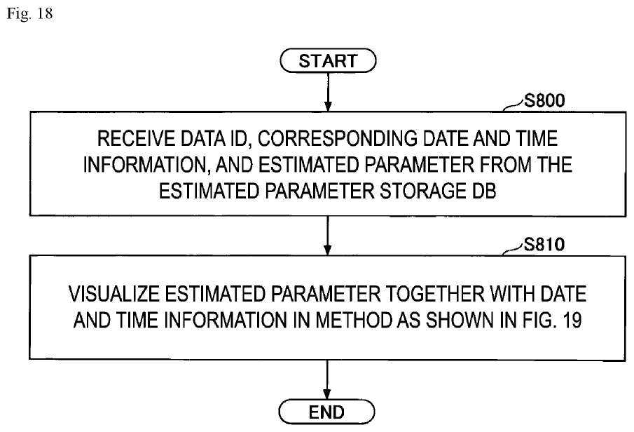

FIG. 18 is a flowchart illustrating operation of the estimation result visualization unit 270. The operation of the estimation result visualization unit 270 will be described with reference to FIG. 18.

### S800

In S800, the estimation result visualization unit 270 receives a data ID, corresponding date and time information, and an estimated parameter from the estimated parameter storage DB 260.

### S810

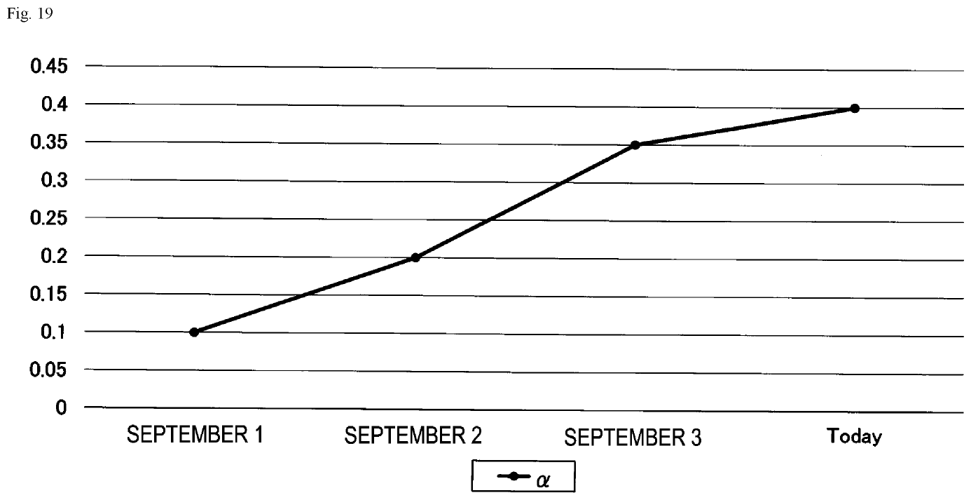

In S810, the estimation result visualization unit 270 visualizes the estimated parameter together with date and time information. For example, by taking date and time information on the horizontal axis and a value of the estimated parameter on the vertical axis, and visualizing it with a line chart for date and time information, degree of change of the estimated parameter with respect to time is visualized. Note that the estimation result visualization unit 270 may be a function unit including a display configured to display to the user, or may be a function unit that creates display information (for example, web page data) to transmit to a terminal. FIG. 19 illustrates a display example of a visualization result.

FIG. 19 is an example of a case where t=T (=4) corresponds to today, t=3 corresponds to September 3, t=2 corresponds to September 2, and t=1 corresponds to September 1, in an estimated parameter indicated by {αₜ}^{T}ₜ₌1. September 3, September 2, September 1, and so on are examples of a past reference point, and behavior on a day where a has a large value has a large impact on a psychological state of today.

Note that while FIG. 19 is an example of displaying a as an estimated parameter, β (for example, {β_{t, 1}}^{T}ₜ₌₁) may be displayed. β also indicates importance at a past reference point for target date and time.

### Effects of Embodiment

The technique according to the present embodiment described above enables estimation of a psychological state felt by the user with high accuracy based on behavior data of the user. That is, it is possible to estimate a past reference point that is effectively suited to the user from behavior data, to estimate a psychological state of the user with high accuracy, and to achieve analysis of a reference point of a psychological state of the user based on a parameter obtained as a result of the estimation. The above is more specifically as described below.

The technique according to the present embodiment not only extracts a feature vector from behavior data, but also trains a model using difference vector data for past date and time, and utilizes an obtained model for psychological state estimation, so that it is possible to estimate a psychological state of the user that has not been able to be estimated in the related art.

Further, by automatically estimating degree of importance of feature vector data and difference vector data effective for estimation of a psychological state of the user by the self-attention mechanism at each time (each date and time), it is possible to estimate a psychological state of the user with high accuracy.

Further, by automatically estimating degree of importance of series data from present to past that is effective for estimation of a psychological state of the user by the self-attention mechanism, it is possible to estimate a psychological state of the user with high accuracy.

Further, by using the self-attention mechanism for estimation of a psychological state of the user, it is possible to estimate different degrees of importance for behavior data depending on the user and a time, and, by visualizing the estimated degree of importance, it is possible to understand information of which date and time the user takes into consideration, and how much difference is evaluated for past date and time as viewed from the present.

### SUMMARY OF EMBODIMENT

The present specification discloses at least a training apparatus, an estimation apparatus, a training method, an estimation method, and a program described in items described below.

### Item 1

A training apparatus including:

a feature extraction unit configured to extract feature vector data from behavior data of each date and time;

a reference point extraction unit configured to perform, for each date and time, processing of calculating a difference between feature vector data of certain date and time and each of one or more pieces of feature vector data in past within a predetermined period from the date and time, and extracting one or more pieces of difference vector data corresponding to the feature vector data of the date and time; and

a state estimation model training unit configured to train a state estimation model using feature vector data of each date and time, difference vector data, and state information.

### Item 2

The training apparatus according to Item 1, in which

the state estimation model is a DNN including a self-attention mechanism that estimates a parameter indicating importance of behavior data of date and time in past for a state of certain date and time.

### Item 3

An estimation apparatus including:

a feature extraction unit configured to extract feature vector data from behavior data of each date and time;

a reference point extraction unit configured to calculate a difference between feature vector data of target date and time for state estimation and each of one or more pieces of feature vector data in past within a predetermined period from the target date and time, and extracting one or more pieces of difference vector data corresponding to the feature vector data of the target date and time; and

a state estimation unit configured to input, to the state estimation model trained by the state estimation model training unit according to Item 1 or 2, the feature vector data of the target date and time, one or more pieces of feature vector data in past within the predetermined period, and the one or more pieces of difference vector data to acquire state information of the target date and time from the state estimation model.

### Item 4

The estimation apparatus according to Item 3, in which

the state estimation unit acquires, from the state estimation model, a parameter indicating importance of date and time in past for a state of the target date and time, and

the estimation apparatus further includes an estimation result visualization unit that displays a value of the parameter in a time series.

### Item 5

A training method executed by a training apparatus, the training method including:

a feature extraction step of extracting feature vector data from behavior data of each date and time;

a reference point extraction step of performing, for each date and time, processing of calculating a difference between feature vector data of certain date and time and each of one or more pieces of feature vector data in past within a predetermined period from the date and time, and

extracting one or more pieces of difference vector data corresponding to the feature vector data of the date and time; and

a state estimation model training step of training a state estimation model using feature vector data of each date and time, difference vector data, and state information.

### Item 6

An estimation method executed by an estimation apparatus, the estimation method including:

a feature extraction step of extracting feature vector data from behavior data of each date and time;

a reference point extraction step of calculating a difference between feature vector data of target date and time for state estimation and each of one or more pieces of feature vector data in past within a predetermined period from the target date and time, and extracting one or more pieces of difference vector data corresponding to the feature vector data of the target date and time; and

a state estimation step of inputting, to the state estimation model trained by the state estimation model training step according to Item 5, the feature vector data of the target date and time, one or more pieces of feature vector data in past within the predetermined period, and the one or more pieces of difference vector data to acquire state information of the target date and time from the state estimation model.

### Item 7

A program for causing a computer to operate as each unit in the training apparatus according to Item 1 or 2.

Although the present embodiment has been described above, the present invention is not limited to such a specific embodiment, and various modifications and changes can be made within the scope of the gist of the present invention described in the claims.

### Reference Signs List

100 Training apparatus

110 Behavior data DB

120 Behavior data preprocessing unit

130 Feature extraction unit

140 Reference point extraction unit

150 Self-evaluation data DB

160 Psychological state estimation model construction unit

170 Psychological state estimation model training unit

180 Psychological state estimation DNN model DB

190 Estimated parameter storage DB

200 Estimation apparatus

210 Behavior data preprocessing unit

220 Feature extraction unit

230 Reference point extraction unit

240 Psychological state estimation DNN model DB

250 Psychological state estimation unit

260 Estimated parameter storage DB

270 Estimation result visualization unit

1000 Drive apparatus

1001 Recording medium

1002 Auxiliary storage apparatus

1003 Memory apparatus

1004 CPU

1005 Interface apparatus

1006 Display apparatus

1007 Input apparatus

1. A training apparatus comprising a processor configured to execute a method comprising:
extracting feature vector data from behavior data of each date and time;
calculating, for at least a date and time, a difference between feature vector data of certain date and time and each of one or more pieces of feature vector data in past within a predetermined period from the date and time;
extracting one or more pieces of difference vector data corresponding to the feature vector data of the date and time; and
training a state estimation model using feature vector data of the at least a date and time, difference vector data, and state information.

2. The training apparatus according to claim 1, wherein
the state estimation model includes a deep neural network with at least a self-attention mechanism that estimates a parameter indicating importance of behavior data of date and time in past for a state of certain date and time.

3. An estimation apparatus comprising a processor configured to execute a method comprising:
extracting feature vector data from behavior data of each date and time;
calculating a difference between feature vector data of target date and time for state estimation and one or more pieces of feature vector data in past within a predetermined period from the target date and time;
extracting one or more pieces of difference vector data corresponding to the feature vector data of the target date and time; and
inputting, to a state estimation model, the feature vector data of the target date and time, one or more pieces of feature vector data in past within the predetermined period, and the one or more pieces of difference vector data to acquire state information of the target date and time from the state estimation model.

4. The estimation apparatus according to claim 3, wherein
the inputting further includes acquiring, from the state estimation model, a parameter indicating importance of date and time in past for a state of the target date and time,
the processor further further configured to execute a method comprising:
displaying a value of the parameter in a time series.

5. A training method executed by a training apparatus, the training method comprising:
extracting feature vector data from behavior data of each date and time;
calculating, for at least a date and time, a difference between feature vector data of certain date and time and each of one or more pieces of feature vector data in past within a predetermined period from the date and time;
extracting one or more pieces of difference vector data corresponding to the feature vector data of the date and time; and
training a state estimation model using feature vector data of at least the date and time, difference vector data, and state information.

6-7. (canceled)

8. The training apparatus according to claim 1, wherein the feature vector data represent at least behavior data of a user for estimating psychological state felt by the user.

9. The training apparatus according to claim 1, wherein the state estimation model estimates a psychological state felt by the user.

10. The training apparatus according to claim 1, wherein the state information is associated with a psychological state, and wherein the psychological state includes at least one of a degree of stress, a degree of health, or a degree of happiness.

11. The training apparatus according to claim 2, wherein the feature vector data represent at least behavior data of a user for estimating psychological state felt by the user.

12. The training apparatus according to claim 3, wherein the feature vector data represent at least behavior data of a user for estimating psychological state felt by the user.

13. The estimation apparatus according to claim 3, wherein the state estimation model estimates a psychological state felt by the user.

14. The estimation apparatus according to claim 3, wherein the state information is associated with a psychological state, and wherein the psychological state includes at least one of a degree of stress, a degree of health, or a degree of happiness.

15. The estimation apparatus according to claim 3, wherein
the state estimation model includes a deep neural network with at least a self-attention mechanism that estimates a parameter indicating importance of behavior data of date and time in past for a state of certain date and time.

16. The estimation apparatus according to claim 15, wherein the feature vector data represent at least behavior data of a user for estimating psychological state felt by the user.

17. The training method according to claim 5, wherein the feature vector data represent at least behavior data of a user for estimating psychological state felt by the user.

18. The training method according to claim 5, wherein the state estimation model estimates a psychological state felt by the user.

19. The training method according to claim 5, wherein the state information is associated with a psychological state, and wherein the psychological state includes at least one of a degree of stress, a degree of health, or a degree of happiness.

20. The training method according to claim 5, wherein
the state estimation model includes a deep neural network with at least a self-attention mechanism that estimates a parameter indicating importance of behavior data of date and time in past for a state of certain date and time.

21. The estimation apparatus according to claim 20, wherein the feature vector data represent at least behavior data of a user for estimating psychological state felt by the user.
